(19) United States
(12) Patent Application Publication
(10) Pub. No.: US 2007/0218152 A1
(43) Pub. Date: Sep. 20, 2007

(54) Process for obtention of decoctions of Vitis labrusca and Vitis vinifera skins

(76) Inventor: Roberto Moura, Rio de Janeiro (BR)

Correspondence Address: ARENT FOX PLLC, 1050 CONNECTICUT AVENUE, N.W., SUITE 400, WASHINGTON, DC, 20036, US

(21) Appl. No.: 11/712,925

(22) Filed: Mar. 2, 2007

Related U.S. Application Data
(62) Division of application No. 10/469,990, filed on May 28, 2004.

(51) Int. Cl. A61K 36/87

(52) U.S. Cl. 424/766

(57) ABSTRACT

The present invention deal with a process to obtain a decoction from Vitis Labrusca and Vitis skins; process to obtain a hydro-alcoholic and hydro-alcoholic-ethyl acetate obtained from the decoction; pharmaceutical preparations containing the decoction and the before mentioned extracts and therapeutic utilization of the preparations and the prevention and treatment of arterial hypertension and other cardiovascular diseases. The decoctions and the extracts, when administered orally to rats with spontaneous arterial hypertension, hypertension induced by chronically inhibition of nitric oxide synthesis or induced by DOCA-salt administration induced a significant reduction of the high levels of arterial blood pressure. Capsules and tablets were prepared with the decoctions and extracts.

### CROSS-REFERENCE TO RELATED APPLICATIONS

This application is a Divisional Application, which claims the benefit of pending U.S. patent application Ser. No. 10/469,990, filed May 28, 2004, which is a national stage application under 35 U.S.C. § 371 of PCT/BR02/00038, filed Mar. 12, 2002. The entire contents of this application are incorporated herein by reference.

### BACKGROUND OF THE INVENTION

Process for obtention of decoctions of Vitis labrusca and Vitis vinifera skins; Process for obtention of hydro-alcoholic and hydro-alcoholic ethyl acetate from the decoctions; pharmaceutical preparations containing the decoction and the extracts and therapeutic indications of the preparations in the prevention and treatment of arterial hypertension and other cardiovascular diseases.

I) Field of Invention

The present invention deal with products that have anti-hypertensive properties, process to obtain products from plants belonging to Vitacea family, more specifically to Vitis labrusca and Vitis vinifera species, application of those products, process to obtain those products, more specifically a process to obtain a decoction from skins of those plants, more specifically a process to obtain an of hydro-alcoholic and hydro-alcoholic-ethyl acetate extracts from those decoctions, more specifically, a process to obtain pharmaceutical preparations containing these products and therapeutic indications of pharmaceutical preparations in the treatment of arterial hypertension and diseases caused by arterial hypertension.

II) Invention Antecedents

Arterial hypertension is a disease with high prevalence among adult population and induces many deleterious effects in hypertensive patients, including cardiac, kidney and cerebral dysfunction. Arterial hypertension is at the moment one of the largest causes of death. Therefore, pharmacological treatment, that have the scope to reduces the high level of arterial blood pressure and the cardiovascular complications from arterial hypertension, is helpful for the patient and supported by health public agency. Usually the pharmacological treatment of hypertension is obtained with use of diuretics, beta blocking agents, inhibitors of the renin-angiotensin system, inhibitors of the sympathetic system and vasodilators compounds.

Data from the literature suggest that cardiovascular mortality is inversely related to moderate ingestion of alcoholic beverage. Leger et al., (Lancet 1:1017-1028, 1979) suggested that daily ingestion of moderate amount of red wine may be responsible for the low incidence of coronary heart disease. This observation was coined as “French Paradox” (Lancet 338:464-486, 1991) since the incidence of coronary heart disease in the some part of France, where the ingestion of a high fat diet is not correlate with a high incidence of coronary heart disease, as observed in other countries. One explanation for the French Paradox could be related to the presence of polyphenols in the red wine, compounds that could acts on the metabolism of LDE, an important risk factor for coronary heart disease.

At the moment the mechanisms of the protection induced by small intake of wine on the reduction on cardiac mortality is not known, but probably an action on the metabolism of lipids may be taken in consideration. However considering that arterial hypertension is an important risk factors for coronary heart disease, the present invention suggest a protective mechanism based on the experimental data object of this patent, that showed that products obtained from grape skins reduce the development of arterial hypertension in rats, and also reduce the high levels of arterial blood pressure in experimental hypertension.

### DETAILED DESCRIPTION OF THE DRAWINGS

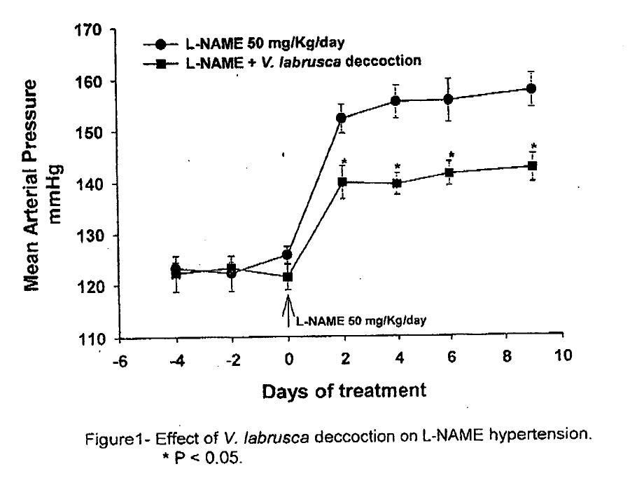

FIG. 1 shows the effect of a decoction of Vitis labrusca on L-NAME hypertension.

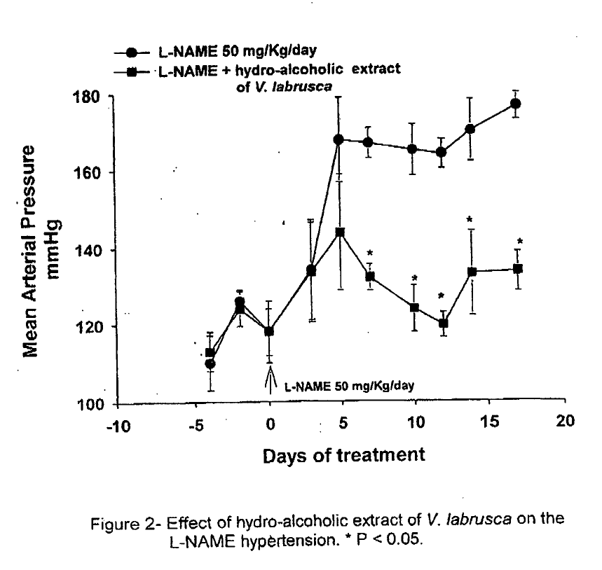

FIG. 2 shows the effect of a hydro-alcoholic extract of Vitis labrusca on L-NAME hypertension.

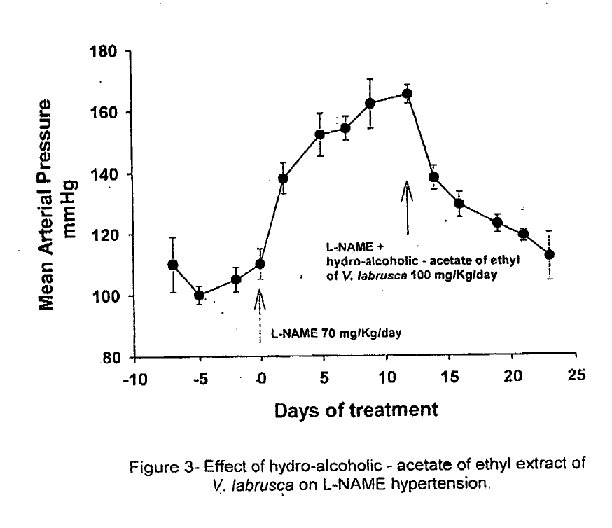

FIG. 3 shows the effect of a hydro-alcoholic-acetate of ethyl extract of Vitis labrusca on L-NAME hypertension.

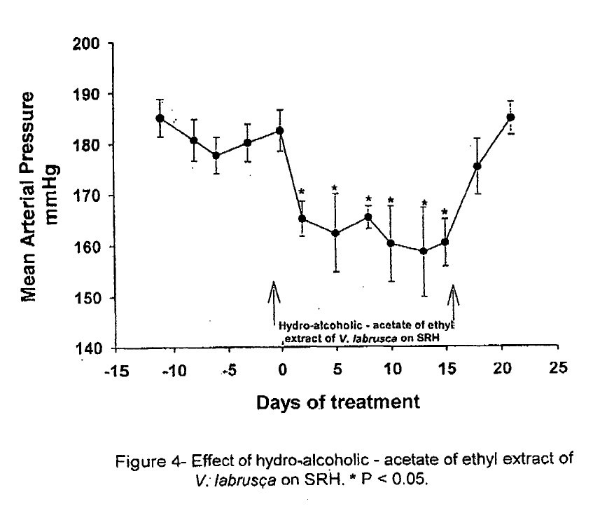

FIG. 4 shows the effect of a hydro-alcoholic-acetate of ethyl extract of Vitis labrusca on SRH.

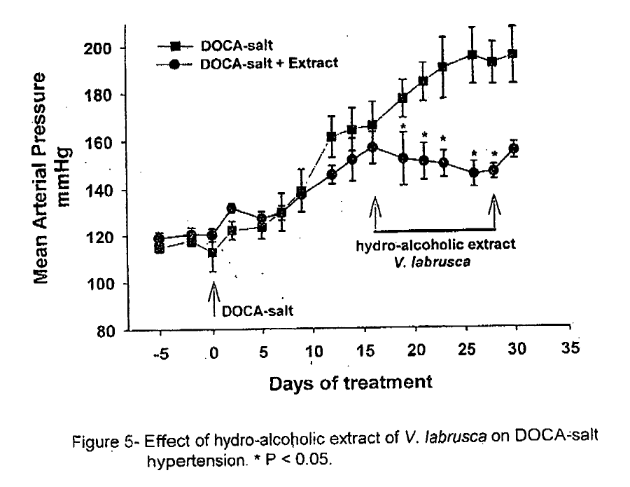

FIG. 5 shows the effect of a hydro-alcoholic extract of Vitis labrusca on DOCA-salt hypertension.

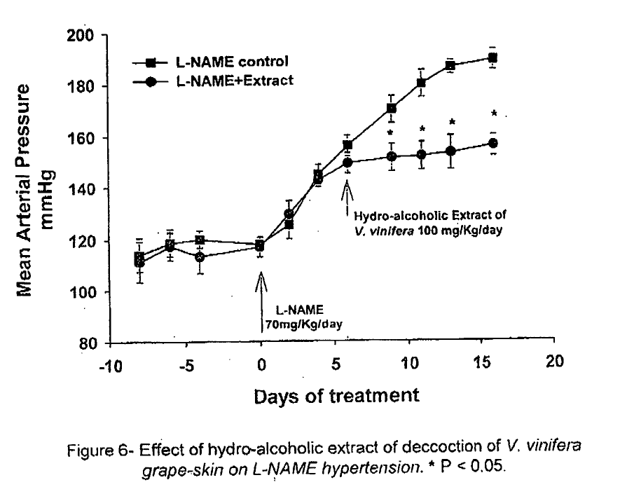

FIG. 6 shows the effect of a hydro-alcoholic extract of a decoction of Vitis vinifera grape skin on L-NAME hypertension.

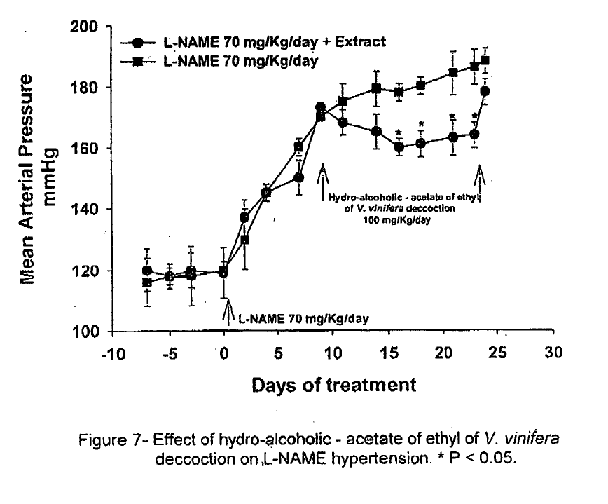

FIG. 7 shows the effect of a hydro-alcoholic-acetate of ethyl extract of Vitis vinifera decoction on L-NAME hypertension.

### III) SUMMARY OF THE INVENTION

The present invention refers to a process to obtain products that have anti-hypertensive properties. Refer also of those products that are obtained from plants belonging to Vitacea family, more specifically Vitis vinifera and Vitis labrusca species, in particular, to a process to obtain products that include extraction of those products from the fruits of those plants.

More particularly, the present invention relates to a process to obtain products that have anti-hypertensive activity, that include separation of skins, pulps and seeds of the fruits, to obtain a decoction from the skins of the fruits and extraction of the decoction with solvents, particularly solvents physiologically acceptable as ethanol, ethylacetate and/or its mixtures, and process the decoctions and extracts to obtain products with pharmacological activities.

More particularly, the present invention also relates to methods to obtain products with anti-hypertensive properties as a decoction, that include an extraction with a solvent physiologically acceptable, as for instance, water as a decoction for 3 to 30 minutes.

Also, the present invention relates to a method for processing the decoctions and extracts to obtain products with pharmacological activities.

Also, the present invention relates to a process to obtain products as hydro-alcoholic and hydro-alcoholic-ethyl acetate extracts, and separation, concentration and lyophilization to obtain the products.

Also, the present invention relates, as mention above, to the before mentioned products per se and also the utilization of the before mentioned products and medicines containing the before mentioned products, with anti-hypertensive properties.

IV) Condensed Description of the Methodology Used.

The present invention is supported by scientific data obtained in chemical and pharmacological experimentations. Below we give a brief view of the investigative process that supports the invention.

IV.A) Method to Obtain the Grape-Skin Decoctions.

Vitis labrusca and Vitis vinifera fruits were washed and after separation from the pulps, the skins were put inside a recipient made of neutral glass or stain-less steel, containing a certain amount of distilled water, boiled, minced and left macerated for a certain period of time, and further filtered in order top obtain the liquid phase of the decoction. The liquid phase is concentrated in a low-pressure rotator evaporator at approximately 40° C. and then lyophilized. The lyophilized is kept frozen (−20° C.).

IV.B) Method to Obtain the Grape-Skin Hydro-Alcoholic Extract from the Skin-Decoction.

The fruits of Vitis labrusca or Vitis vinifera were washed and after separation from the pulps, the skins were put inside recipient made of neutral glass or stainless-steel containing a certain amount of destined water and boiled. The decoction was minced, extract with ethanol, same amount of water then left macerated for a certain period of time. The mixture is filtered, the liquid phase is kept refrigerated and the semi-solid phase can be again extract with a mixture of water/ethanol (v:v) for one or two time. The liquid phases are concentrated in a low-pressure rotator evaporator at approximately 40 to 60° C. and then lyophilized and kept at −20° C.

IV.C) Method to Obtain Grape-Skin Hydro-Alcoholic-Ethyl Acetate Extract from the Skin-Decoction.

The fruits of Vitis labrusca or Vitis vinifera were washed and after separation from the pulps, the skins were put inside recipient made of neutral glass or stainless-steel containing a certain amount of destined water, boiled, minced, extract with a mixture of ethanol-ethyl acetate-decoction (v:v:v) and then left macerated for a certain period of time. The mixture is filtered, the liquid phase is kept refrigerated and the semi-solid phase can be again extract with a mixture of water/ethanol/acetate ethyl (v:v:v) for one or two time, and the liquid phases are concentrated in a low pressure rotator evaporator at approximately 40 to 60° C. and then lyophilized and kept at −20° C.

IV.D) Pharmacodynamic and Pharmacotechnical Methods.

The pharmacological activities of the products obtained from Vitis labrusca and Vitis vinifera-skins were assessed by pharmacodynamic tests that assessed the anti-hypertensive rats and L-NAME hypertensive rats. The pharmacotechnical method refers the way to obtain capsules containing the lyophilized of the extracts obtained from Vitis labrusca and Vitis vinifera skins.

### V.) DETAILED DESCRIPTION OF THE INVENTION

In the ambit of the present invention, the fruits of Vitis labrusca and Vitis vinifera species, before been submitted to the process of extraction, according to the invention, if not utilized after the harvest, can be stored for long periods at the temperature from +4 to −20° C.

V.A) Method to Obtain the Grape-Skin Decoction.

Fruits of Vitis labrusca and Vitis vinifera are washed in water, and the skin is separated from the pulp. The skins are washed in water and later put inside of neutral glass or stainless steel recipient in the proportion of 1 to 100 g of skin to 100 ml of water, for instance 25 g/100 ml and boiled for 3 to 60 minutes, for instance about 5 minutes. After the time of boiling, the decoction is left to cool to 20 to 90° C., for instance 75° C. and minced. The minced decoction was macerated for a certain period of time (1 hour to 30 days) for instance 6 hours inside a refrigerate at 4° C. or at room temperature for instance 25° C. under agitation. The decoction is filtered in a sieve with 0.1 to 1.0-mm pore, for instance 0.2 mm, being also filtered through gauze and finally filtered through a paper filter Whartman n.1. The liquid phase is concentrated in a low-pressure evaporator at a temperature of 30 to 60° C. for instance 40° C. and then lyophilized and kept under −4 to −70° C.

V.B) Method to Obtain the Grape-Skin Hydro-Alcoholic Extract from the Decoction.

The grape-skin decoction, after been minced short after boiling, is extracted with ethanol 95% in a proportion of decoction/ethanol (v:v) of 1:0.5 to 1:10, for instance 1:1. That mixture is minced and macerated for a certain period of time of 3 hours to 30 days, for instance 6 hours and kept inside a refrigerator at 4° C. or at room temperature at 25° C. and shaken. At the end of the maceration period it is filtered through a sieve with 0.1 to 1 mm pores, for instance 0.2 mm, being also filtered through gauze and finally filtered through a filter paper, Whartman n.l. The semi-solid phase can be extracted again in the same conditions as described above for 1 or 3 times, for instance 2 times. The liquid phase of the first extraction is kept inside a refrigerator at 4° C. and then added to the liquid phase of the other extractions. The final liquid phase is concentrated under low pressure evaporator at a temperature of 35 to 65° C., for instance 40° C. e then lyophilized and kept at −4° C. to −70° C., for instance −20° C.

V.C) Method to Obtain the Grape-Skin Hydro-Alcoholic-Acetate of Ethyl Extract from the Decoction.

The decoction, after been minced after boiling, is extracted with a mixture of decoction/ethanol/ethyl acetate (v:v:v) of variable proportion for instance 1:1:1. That mixture is minced and macerated for a certain period of time of 3 hours to 30 days, for instance 6 hours and kept inside a refrigerator at 4° C. or at room temperature at 25° C. and shaken. At the end of the maceration period the extract is filtered through a filter paper Whartman n.1 and the liquid phase kept inside a refrigerator at 4° C. and the semi-solid phase can be again extracted at the same condition as described above for one or three times, for instance two times. The liquid phrase of the first extraction is kept inside a refrigerator at 4° C. and then added to the liquid phase of the other extractions. The final liquid phase is concentrated under low pressure evaporator at a temperature of 35 to 65° C., for instance 40° C. and then lyophilized and kept at 4 C to −70° C., for instance −20° C.

VI.) General Observation

The specific procedure described in item V.A. to V.C above, were described not with the scope to limit, but with the scope to illustrate the possibilities of realization of the present invention, with ambit is limited only by the claim annex.

VII.) Pharmacodynamical and Pharmacotechnical Methods

The pharmacological activities of the various products obtained from the Vitis labrusca and Vitis vinifera skins were assessed by pharmacodynamic methods that study the anti-hypertensive activity of the products in the following models of experimental hypertension: DOCA-salt hypertension, spontaneous hypertensive rats (SHR) and hypertension induced by inhibition of nitric oxide synthase. The pharmacotechnical method refer to the method to obtain capsules containing the lyophilized residue of the products obtained from Vitis labrusca and Vitis vinifera skins.

### VIII) ILLUSTRATIVE EXAMPLES OF THE INVENTION

Below are examples with the objective to illustrate and not to limit the present invention, which purpose, as mention above, has its delimitation's in the claim annex.

VIII.A) Method to Obtain the Lyophilized of the Vitis labrusca Grape Skin Decoction.

Approximately 2 000 g of Vitis labrusca fruits were washed in tap water. The skins were isolated from the pulps and washed in tap water during approximately 3 minutes. The skin (1000 g) were boiled in 4000 mL of distilled water for 5 minutes. After boiling, while the decoction was still warm, the decoction was minced at approximately 80° C. and left for maceration for 6 hours under shaking. The decoction was filtered through a sieve with 0.2 mm pores and then filtered in gauze and then through a filter paper Whartman n.1. The liquid phase, obtained after filtration was concentrated under low pressure evaporator at 45° C., lyophilized and kept at −20° C., until the day of use.

VIII.B) Method to Obtain the Lyophilized of the Vitis vinifera Grape-Skin Decoction.

Approximately 2 000 g of Vitis vinifera fruits were washed in tap water. The skins were isolated from the pulps and washed in tap water during approximately 3 minutes. The skin (1000 g) were boiled in 400 mL of distilled water for 5 minutes. After boiling, while the decoction was still warm, the decoction was minced at approximately 80° C. and left for maceration for 6 hours under shaking. The decoction was filtered through a sieve with 0.2 mm pores and then filtered in gauze and then through a filter paper Whartman n.1. The liquid phase, obtained after filtration was concentrated under low-pressure evaporator at 45° C., lyophilized and kept at −20° C., until the day of use.

VIII.C) Method to Obtain the Lyophilized of the Hydro-Alcoholic Extract Obtained from Vitis labrusca Grape-Skin Decoction.

Approximately 1 000 g of Vitis labrusca fruits were washed in tap water. The skins were isolated from the pulps and washed in tap water during approximately 3 minutes. The skin (500 g) were boiled in 2 L of distilled water for 5 minutes. After boiling, the decoction was minced. Two liter of ethanol was added to the decoction and minced. The extracted was left macerating for 6 hours and filtered through a sieve, pores 0.2-mm and then filtered through a filter paper Whartman n.1. The liquid phase, obtained after filtration was concentrated under low-pressure evaporator at 45° C., to evaporate the ethanol and then lyophilized to obtain the lyophilized of the hydro-alcoholic extract of the Vitis labrusca decoction.

VIII.D) Method to Obtain the Lyophilized of the Hydro-Alcoholic Extract Obtained from Vitis vinifera Grape-Skin Decoction.

Approximately 1 000 g of Vitis vinifera fruits were washed in tap water. The skins were isolated from the pulps and washed in tap water during approximately 3 minutes. The skins (500 g) were boiled in 2 L of distilled water for 5 minutes. After boiling, the decoction was minced. Two liters of ethanol was added to the decoction and minced. The extracted was left macerating for 6 hours and filtered through a sieve, pores 0.2-mm and then filtered in a filter paper Whartman n.1. The liquid phase, obtained after filtration was concentrated under low-pressure evaporator at 45° C., to evaporate the ethanol and then lyophilized to obtain the lyophilized hydro-alcoholic extract of the Vitis vinifera decoction.

VIII.E) Method to Obtain the Lyophilized of the Hydro-Alcoholic-Ethyl Acetate Extract Obtained from Vitis labrusca Grape-Skin Decoction.

Approximately 1 000 g of Vitis labrusca fruits were washed in tap water. The skin (500 g) were isolated from the pulps and washed in tap water during approximately 3 minutes. The skins were boiled in 2 L of distilled water for 5 minutes. After boiling, the decoction was minced. One liter of ethanol plus one liter of ethyl acetate were added to the decoction and minced. The extracted was left macerating for 6 hours and filtered through a sieve, pores 0.2-mm and then filtered in a filter paper Whartman n.1. The liquid phase, obtained after filtration was concentrated under low-pressure evaporator at 45° C., to evaporate the ethanol and then lyophilized to obtain the lyophilized hydro-alcoholic extract of the Vitis labrusca decoction.

VIII.F) Method to Obtain the Lyophilized of the Hydro-Alcoholic-Ethyl Acetate Extract Obtain from Vitis vinifera Grape-Skin Decoction.

Approximately 1 000 g of Vitis vinifera fruits were washed in tap water. The skins were isolated from the pulps and washed in tap water during approximately 3 minutes. The skins were boiled in 2 L of distilled water for 5 minutes. After boiling, the decoction was minced. One liter of ethanol plus one liter of ethyl acetate were added to the decoction and minced. The extracted was left macerating for 6 hours and filtered through a sieve, pores 0.2-mm and then filtered in a filter paper Whartman n.1. The liquid phase, obtained after filtration was concentrated under low-pressure evaporator at 45° C., to evaporate the ethanol and then lyophilized to obtain the lyophilized hydro-alcoholic extract of the Vitis vinifera decoction.

IX.) Example of Biological Tests Performed with the Products of the Invention.

The anti-hypertensive activity of lyophilized from various products was access by testing its efficacy of the lyophilized to reduce the levels of experimental arterial hypertension and to reduce the development of hypertension in rats. The anti-hypertensive activity was accessed in adult male Wistar rats, spontaneous hypertensive or made hypertensive by the following methods: nitric oxide inhibition by use of an analogue of L-arginine, that is, L-NAME, and subcutaneous injection of DOCA followed by orally administration of saline in uninephrectomized rats.

Arterial blood pressure was measured in the tail of rats by a noninvasive method while the rats were awake, using a cuff and a sensor c connected to equipment manufactured by Letica-Barcelona-Spain. The lyophilized was administrated orally, in the drinking water, so that the rats were treated with the products continuously during the period of treatment. Arterial pressure was measured three times per week before the and during the treatment with the lyophilized. The values of arterial blood pressure were compared using Student's tests and the difference were considered significantly when p<0.05.

IX.A) Effects of the Lyophilized Obtained from the Decoction of Vitis labrusca Skins in the Arterial Hypertension Induced by Inhibition of Nitric Oxide Synthesis in Rats.

Adult, male Wistar rats (250-350 g, n=10) were kept in a cage with no more than 4 animals per cage. Mean arterial blood pressure was measured noninvasively, by Letica (Barcelona) equipment. A cuff and a sensor around the tail of the animal were connected to the equipment. The cuff is inflated automatically in order the measure the mean arterial blood pressure.

After a period of adaptation the experimental conditions, for measurement the mean arterial blood pressure, the pharmacodynamical test was started. During the adaptation period, body weight was estimated three times a week, the animals received tap water and food at libitum, and the daily intake of water was estimated.

The animals were divided in two groups of 5 rats. One group (control) was treated orally with L-NAME, 50 mg/kg/day, diluted in the drinking water. The other group was also treated with L-NAME, 50 mg/kg/day plus 100 mg/kg/day of the lyophilized of the decoction of the decoction of Vitis labrusca in the drinking water. FIG. 1 shows the anti-hypertensive effect of the lyophilized of the decoction of Vitis labrusca in this particular experiment.

IX.B) Effects of the Lyophilized of the Hydro-Alcoholic Extract Obtained from the Decoction of Vitis labrusca Skins in the Arterial Hypertension Induced by Inhibition of Nitric Oxide Synthesis in Rats.

Adult, male Wistar rats (250-350 g, n=12) were kept cage with no more than 4 animals per cage. Mean arterial blood pressure was measured noninvasively, by Letica (Barcelona) equipment. A cuff and a sensor around the tail of the animal were connected to the equipment. The cuff is inflated automatically in order the measure the mean arterial blood pressure.

After a period of adaptation the experimental conditions, for measurement the mean arterial blood pressure, the pharmacodynamical test was started. During the adaptation period, body weight was estimated three times a week, the animals received tap water and food at libitum, and the daily intake of water was estimated.

The animals were divided in two groups of 6 rats. One group (control) was treated orally with L-NAME, 50 mg/kg/day, diluted in the drinking water. The other group was also treated with L-NAME, 50 mg/kg/day plus 100 mg/kg/day of the lyophilized of the hydro-alcoholic extract of decoction of Vitis labrusca in the drinking water, five days after the beginning of treatment with L-NAME. FIG. 2 shows the anti-hypertensive effect of the lyophilized of the hydro-alcoholic extract obtained from the decoction of Vitis labrusca in this particular experiment.

IX.C) Effects of the Lyophilized of the Hydro-Alcoholic-Acetate of Ethyl Extract Obtained from the Decoction of Vitis labrusca Skins in the Arterial Hypertension Induced by Inhibition of Nitric Oxide Synthesis in Rats.

Adult, male Wistar rats (250-350 g, n=12) were kept cage with no more than 4 animals per cage. Mean arterial blood pressure was measured noninvasively, by Letica (Barcelona) equipment. A cuff and a sensor around the tail of the animal were connected to the equipment. The cuff is inflated automatically in order the measure the mean arterial blood pressure.

During the adaptation period, the animal received food and water “as libitum” and the daily intake of water was estimated. Body weight was estimated three times a week. After the levels of basal pressure were obtained, the rats were treated with L-NAME 70 mg/kg/day in the drinking water. Once the arterial pressure reached elevated level, the animal was treated with 100 mg/kg/day of the lyophilized of the hydro-alcoholic-ethyl acetate plus L-NAME. As can been observed in FIG. 3, the extract induced a significant anti-hypertensive effect in this particular experiment.

IX.D) Effects of the Lyophilized of the Hydro-Alcoholic-Ethyl Acetate Extract Obtained from the Decoction of Vitis labrusca Skin in Rats with Spontaneous Hypertension.

The antihypertensive activity of hydro-alcoholic-ethyl acetate extract obtained from the decoction of Vitis labrusca was accessed in five spontaneous hypertensive rats weighting 250 to 305 g. Before the beginning of the treatment with the extract the arterial pressure was measured during two weeks, when after that period, the animals were treated with the extract induced a significant anti-hypertensive response in this particular kind of experimental hypertension.

IX.E) Effects of the Lyophilized of the Hydro-Alcoholic Extract Obtained from the Decoction of Vitis labrusca Skins in Rats with DOCA-Salt Hypertension.

Twelve Wistar male rats, weighting 250 to 350 g were divided in two groups, each group kept in different cages. All rats were uninephrectomized. Arterial pressure was measured non invasively using a Letica (Barcelona) equipment, that include a cuff, a sensor, put around the rat tail and connected to the equipment that inflate automatically the cuff and therefore measure the level of mean arterial pressure. Seven days after the nephrectomy, the animal is adapted to the pressure measurement procedure and received food and water “ad libitum” and body weight was estimated three times a week.

After a period of adaptation the animals were treated subcutaneously with 25 mg/kg/week and received saline as drinking water. Arterial pressure was measured three times a week. After the beginning of the treatment, the arterial blood pressure started to elevate. Sixteen days after the beginning of treatment, when the arterial blood pressure had reached a high level, 6 rats were treated orally with the extract 100 mg/kg/day. The other group of 6 rats were treated with only DOCA plus saline. As shown in FIG. 5, the extract induced a significant reduction of the mean arterial blood pressure in hypertensive rats.

IX.F) Effects of the Lyophilized Obtained from the Decoction of Vitis vinifera Skins in the Arterial Hypertension Induced by Inhibition of Nitric Oxide Synthesis in Rats.

Adult, male Wistar rats (250-350 g, n=11) were kept cage with no more than 4 animals per cage. Mean arterial blood pressure was measured noninvasively, by Letica (Barcelona) equipment. A cuff and a sensor placed around the tail of the animal were connected to the equipment. The cuff is inflated automatically in order the measure the mean arterial blood pressure.

The animals were submitted to a period of adaptation of the experimental conditions, for measurement the mean arterial blood pressure. During the adaptation period, the animals received tap water and food “ad libitum”, and the daily intake of water was estimated. Body weight was estimated three times a week.

After the basal pressure levels are obtained, one group (control, n=5) was treated orally with 70 mg/kg/day L-NAME in drinking water. The other group (n=6) was also treated with the same dose of L-NAME plus 100 mg/kg/day of lyophilized hydro-alcoholic extract obtained from the decoction of Vitis vinifera skin in the drinking water. As shown in FIG. 6, the extract induced a significant anti-hypertensive effect in this particular experiment.

IX.G) Effects of the Lyophilized of the Hydro-Alcoholic-Ethyl Acetate Extract Obtained from the Decoction of Vitis vinifera Skins in the Arterial Hypertension Induced by Inhibition of Nitric Oxide Synthesis in Rats.

Adult, male Wistar rats (250-350 g, n=12) were kept cage with no more than 4 animals per cage. Mean arterial blood pressure was measured noninvasively, by Letica (Barcelona) equipment. A cuff and a sensor placed around the tail of the animal were connected to the equipment. The cuff is inflated automatically in order the measure the mean arterial blood pressure.

The animals were divided in two groups, were submitted to a period of adaptation of the experimental conditions, for measurement the mean arterial blood pressure. During the adaptation period, the animals received tap water and food “ad libitum”, and the daily intake of water was estimated. Body weight was estimated three times a week.

After the basal arterial pressure levels are obtained, both groups were treated orally with 70 mg/kg/day L-NAME treatment, one group (n=5) received only L-NAME and the other group (n=6) was treated with L-NAME plus the extract, 100 mg/kg/day orally. This treatment induced a significant reduction of the high levels of arterial pressure, when compared with the control group, as shown in FIG. 7.

X) Pharmacotechnical Aspects of the Preparation of Capsules and Tablets Containing the Dry Residue of the Various Fractions Obtained from Skin of the Fruits of Vitis labrusca and Vitis vinifera.

The capsules and/or tablets containing 100 to 500 mg of lyophilized of Vitis labrusca or Vitis vinifera were obtained according to the usual pharmacotechnical procedures. The capsules were obtained in order to contain 100 to 500 mg, for instance 250 mg of the lyophilized plus cornstarch and colloidal silicon dioxide. Each capsule could have the following composition.

| | | | |
|---|---|---|---|
| Lyophilized | 250 | mg | 55.5% |
| Corn starch | 200 | mg | 44.4% |
| Colloidal silicon dioxide | 0.5 | mg | 0.1% |
| Total | 450.5 | mg | 100% |

Cornstarch was added to complete the total mass of the capsule to approximately 450 mg. Colloidal silicon dioxide was used to adsorb humidity and to facilitate the preparation of the capsules.

1-8. (canceled)

9. Process to obtain a hydro-alcoholic-acetate of ethyl extract from a decoction of skins of Vitis labrusca and Vitis vinifera, characterized by the following steps:
a) Separate the fruit into skins and pulps;
b) Wash the skin obtained in step (a) in tap water for 3 minutes.
c) Submit the skins of step (b) to a process of extraction in boiling water for 3 to 10 minutes to obtain the decoction;
d) Mince the decoction of step (c) with a mince during 3 to 5 minutes and macerate during 6 hours to 10 days at room temperature or at 4° C.;
e) Filter the macerate obtained in step (d) in a sieve with 0.2 to 1 mm pores and subsequently in a filter paper;
f) Concentrate the liquid phase obtained in step (e) in a rotator evaporator at low pressure and at a temperature of 35 to 50° C.;
g) Lyophilize the concentrated liquid phase in order to obtain a lyophilized of the decoction with pharmacological activities;
said process characterized by the reason that in addition the decoction obtained in step (c) of claim 1 is extracted with the solvents ethanol and acetate of ethyl in the proportion of 1:1:1, v;v;v and minced strongly during 3 to 5 minutes and then macerated for a period of 6 hours to 10 days at room temperature or at 4° C. to obtain the hydro-alcoholic-acetate of ethyl extract of the decoction.

10. Process according to claim 9 characterized by the reason that the macerated obtained is filtered in sieve with 0.2 to 1.0 mm pores and then in filter paper.

11. Process according to claim 9 characterized by the reason that the liquid phase is concentrated in rotator evaporator under low pressure and at 35 to 50° C.

12. Process according to claim 11 characterized by the reason that the liquid phase after being concentrated is lyophilized to obtain the lyophilized of the hydro-alcoholic-acetate of ethyl extract of the decoction with pharmacodynamical activity.

13-14. (canceled)

15. Pharmaceutical preparations characterized by the reason that contain 200 to 500 mg of lyophilized of the hydro-alcoholic-acetate of ethyl extract obtained from the decoction of Vitis labrusca or Vitis vinifera and in addition 200 mg of corn amide and 0.5 mg of colloidal silicon dioxide.

16. A method of preventing and treating arterial hypertension, comprising administering to a subject in need thereof the pharmaceutical preparations according to claim 15.

17. The method of claim 16, wherein the pharmaceutical preparation is administered to a subject selected from the group consisting of human subjects and animal subjects.
